(19) United States
(12) Patent Application Publication
(10) Pub. No.: US 2021/0045906 A1
(43) Pub. Date: Feb. 18, 2021

(54) DEVICE FOR INCREASING PERFORMANCE, COMFORT AND/OR PREVENTING INJURY OF A USER OF A COMPUTER MOUSE

(71) Applicant: Flashe Gaming Group AB, Linköping (SE)

(72) Inventors: Oskar Ödmark, Linköping (SE); Johannes Sjödin, Nykil (SE)

(21) Appl. No.: 16/969,329

(22) PCT Filed: Feb. 13, 2019
(86) PCT No.: PCT/EP2019/053492

§ 371 Date: Aug. 12, 2020

(30) Foreign Application Priority Data
Feb. 13, 2018 (SE) 1850153-6
Jun. 15, 2018 (SE) 1850735-0
Oct. 15, 2018 (SE) 1851256-6

(51) Int. Cl. A61F 5/01; A63F 13/24

(52) U.S. Cl. CPC A61F 5/013; A61F 2005/0179; A63F 13/24

(57) ABSTRACT

The present disclosure provides a wearable device worn on the forearm of a user, whereby the device provides an ergonomically correct positioning of the forearm and hand for increasing the performance and avoid injuries of a user working with a computer mouse or the like. In particular, the disclosure provides a device for increasing performance, comfort and/or preventing injury of a user of a computer mouse or the like. The device comprises a wrist support (20), having an upper side (21) adapted for receiving the user's wrist and/or lower forearm and an underside (22) adapted to bear against a planar surface, such as a table, wherein the underside (22) presents a bearing arrangement (121a, 121b, 121c, 121d, 121e) for reducing friction against the planar surface, and a hand support (20), adapted for receiving a part of the user's hand. The hand support (20) is horizontally +/−40 degrees, preferably +/−20 degrees or +/−10 degrees, movable relative to the wrist support (10).

### TECHNICAL FIELD

The present disclosure relates to a wearable device for increasing performance, comfort and/or preventing injury of a user of a computer mouse, or the like. The device may be used as a gaming glove, i.e. a glove that is intended for use in computer gaming, or as an ergonomic implement intended for use by any computer operator.

### BACKGROUND

Many computer games are controlled at least partially by a computer mouse. Especially in so-called first-person-shooter games, the mouse may be used to control the aim of the player's weapon. Such control may require a large range of movements, from minute ones to relatively large ones. Moreover, such movements often need to be performed very quickly, leading to a very high movement speed.

It is well known, that use of computer mice may lead to injuries, such as carpal tunnel syndrome, and the like. Various devices have been produced with the aim to reduce the risks associated with the use of computer mice.

U.S. Pat. No. 5,925,007A discloses a carpal cuff, which is designed to reduce the risk of injuries on users of computer mice. Similar devices are disclosed in U.S. Pat. No. 6,082,684A, WO0236050A1 and WO09059492A1.

There is a need for further improvements of these devices.

### SUMMARY

An object of the present disclosure is to provide an improved device for reducing the risk of injury, as well as for improving the performance and/or comfort, of computer mouse users.

The invention is defined by the appended independent claims, with embodiments being set forth in the appended dependent claims, in the following description and in the drawings.

According to a first aspect, there is provided a device for increasing performance, comfort and/or preventing injury of a user of a computer mouse or the like. The device comprises a wrist support, having an upper side adapted for receiving the user's wrist and/or lower forearm and an underside adapted to bear against a planar surface, such as a table. The underside presents a bearing arrangement for reducing friction against the planar surface. The device further comprises a hand support, adapted for receiving a part of the user's hand, wherein the hand support is horizontally +/−40 degrees, preferably +/−20 degrees or +/−10 degrees, movable relative to the wrist support.

The bearing arrangement for reducing friction against the planar surface may be embodied as a roller bearing (e.g. ball transfer units) or as a friction bearing, e.g. using one or more low friction polymer coated support surface.

The device may generally be referred to as a “glove”, which in the present context implies a device that may be applied to the user's hand, wrist and/or forearm.

The hand support may be movably connected to the wrist support.

In the device, movement between the hand support and the wrist support may be limited.

The connection may comprise interacting surfaces on the hand support and on the wrist support, at least one of the surfaces comprising a second bearing arrangement, such as a ball bearing or a slide bearing.

The connection may comprise a rotatable connection by which the hand support is rotatably connected to the wrist support, such that the hand support and the wrist support are rotatable relative to each other about an axis that provides an angle of less than 40 degrees, preferably less than 30 degrees or less than 15 degrees, relative to a vertical direction, when the device is placed on a horizontal surface.

The hand support may be connected to the wrist support by a flexible element, which may be fixedly connected or connectable to each of the hand support and the wrist support.

The flexible element may comprise a pair of attachment portions, connectable to a respective one of the hand support and the wrist support and a waist portion, whereby the attachment portions are movable relative to each other through bending about the waist portion.

The flexible element may present less rigidity in a direction of said bending about the waist portion than in a direction perpendicular thereto, such that the flexible element is easier to bend about an axis (A) that provides an angle of less than 40 degrees, preferably less than 20 degrees or less than 10 degrees, relative to a vertical direction, than about an axis that is horizontal.

A biasing element may be provided to bias the hand support and the wrist support towards a predetermined relative position.

The hand support may be connected to the wrist support by means of a joint which is provided at a position below the user's hand.

Alternatively, or as a supplement, the hand support is connected to the wrist support by means of a joint which is provided at a position above the user's hand.

A device as defined above provides improved performance with respect to smaller sideways movements that the user carries out by merely moving the hand, and not the wrist, relative to the planar surface.

Furthermore, as it reduces the resistance to be overcome when making sideways movements by the hand and/or lower arm, the device reduces the risk of injuries to the shoulder, arm and hand regions, or it alleviates such injuries which have already arisen or begun to arise.

The bearing arrangement may comprise at least one ball bearing, configured such that a spherical surface of the ball bearing protrudes from the underside such that it forms a lowermost portion of the wrist support.

The bearing arrangement may comprise a plurality of ball bearings, positioned such that they define a two-dimensional support area.

Alternatively, or as a supplement, the bearing arrangement may comprise a friction reducing surface, arranged on the underside of the wrist support, and optionally also on an underside of the hand support. Such friction reducing surface may be provided so as to reduce friction against the support on which the computer mouse is operated.

The rotatable connection may comprise a roller bearing.

The device may further comprise a wrist fastening device, for securing the wrist and/or lower forearm to the wrist support.

The wrist fastening device may comprise one or more straps, a cuff or a wristlet.

The device may further comprise a hand frame, connected to the hand support, extending upwardly and/or forwardly and configured to at least laterally interact with the hand.

The hand frame provides added leverage in transferring the hand's movement to the hand support.

The device may further comprise a hand fastening device, for securing the hand to the hand support.

The hand fastening device may comprise a glove, one or more straps, a lacing arrangement, a cuff and/or a wristlet.

The wrist support may be sized to extend over at least 60% of the user's radius bone, preferably at least 70%, at least 80% or at least 90%.

The device may further include a wrist positioning element, comprising a support surface that is shaped so as to position a distal portion of the user's radius bone at a higher vertical level than a proximal portion of the user's radius bone.

According to a second aspect, there is provided a kit of parts comprising a device as described above, and a sleeve, adapted for being applied to a wrist or forearm of the user.

The sleeve may be a compression sleeve, adapted to provide a compressive pressure of 15-60 mmHg on at least a portion of the user's wrist or forearm.

The sleeve may be attached or attachable to the device.

According to a third aspect, there is provided a method of applying a device for increasing performance, comfort and/or preventing injury of a user of a computer mouse or the like. The method comprises providing a device as described above, arranging a lower portion of a ventral side of the user's wrist and/or lower forearm to bear against the upper side of the wrist support, and arranging a portion of the user's hand comprising the carpus bones to bear against an upper side of the hand support.

The method may further comprise attaching the user's wrist and/or lower forearm to the wrist support by means of a wrist fastening device, and optionally attaching the user's hand to the hand support by means of a hand fastening device.

The method may further comprise applying a compression sleeve to the user's wrist or forearm prior to arranging the lower portion of the ventral side of the user's wrist to bear on the upper side of the wrist support.

According to a fourth aspect, there is provided use of a device as described above, for computer gaming, preferably for a battle game, such as a first-person shooter type game or a multiplayer online battle arena type game. Hence, the device is suitable for use in first-person shooter type games, such as Counter-Strike: Global Offensive®, since it reduces friction against the support surface (such as a table) for long sideways movements. Moreover, the device is suitable for online battle arena type games, such as League of Legends®, since it also allows for reduced friction when making small sideways movements.

### BRIEF DESCRIPTION OF THE DRAWINGS

FIG. 1a is a schematic perspective view of a gaming glove.

FIG. 1b is a schematic side view of the gaming glove in FIG. 1a.

FIG. 2a is a schematic top view of the gaming glove in FIG. 1a.

FIG. 2b-2d are schematic sectional views of the gaming glove in FIG. 2a.

FIG. 3 schematically illustrates a first joint concept for providing a rotatable joint between the wrist support and the hand support.

FIG. 4 schematically illustrates a second joint concept.

FIGS. 5a-5b schematically illustrates a third joint concept.

FIG. 6 schematically illustrates a fourth joint concept.

FIG. 7 schematically illustrates a fifth joint concept.

FIG. 8 schematically illustrates a sixth joint concept.

FIG. 9 schematically illustrates an embodiment of a gaming glove worn by a user, as seen from below.

FIG. 10 schematically illustrates an embodiment of a gaming glove worn by a user, as seen from above.

FIGS. 11a-11b schematically illustrate an ergonomic support member, which may be incorporated into the gaming glove.

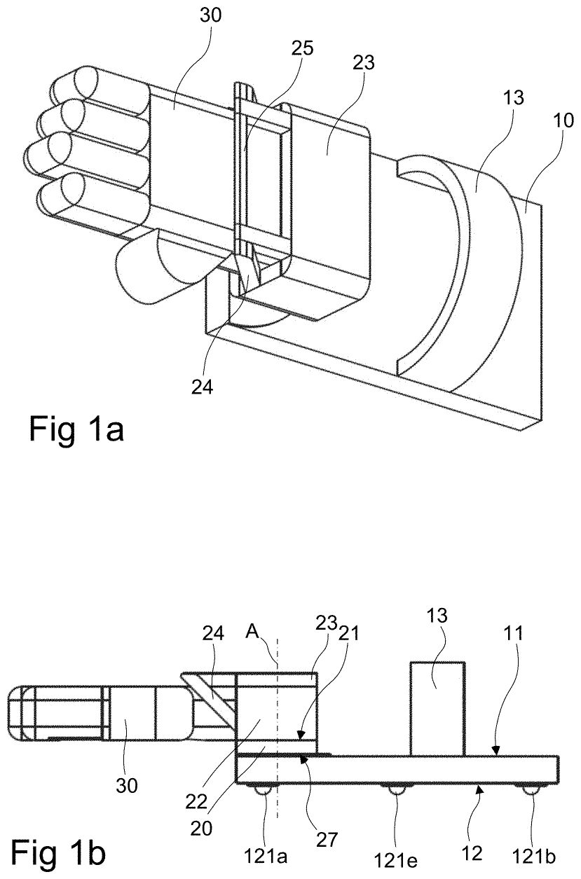

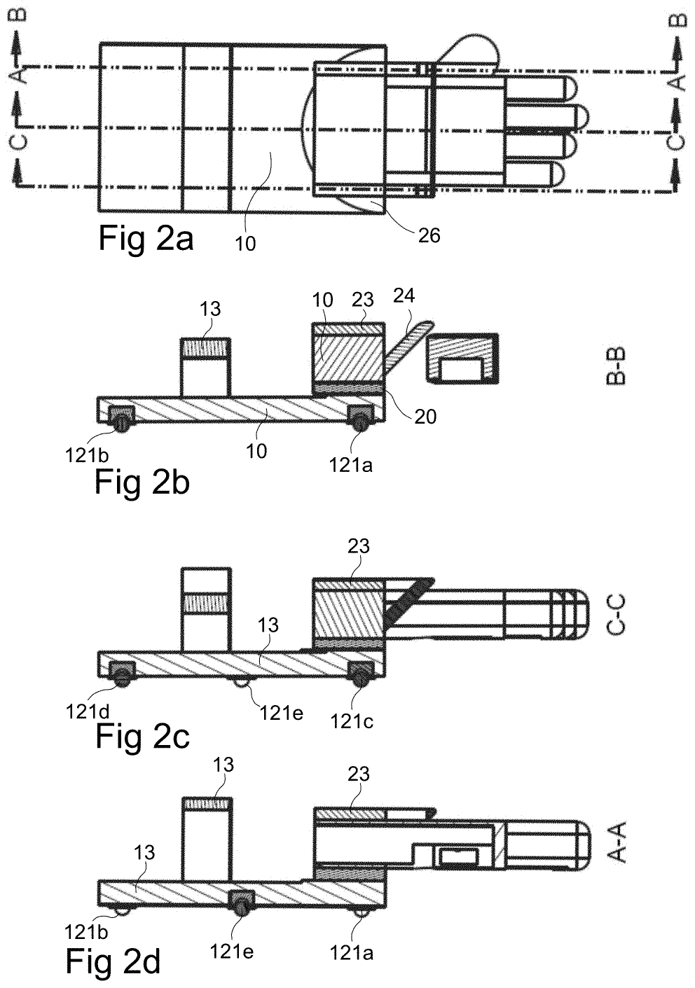

### DETAILED DESCRIPTION

Referring to the drawings, a gaming glove will according to the present disclosure will now be described.

The gaming glove comprises a wrist support 10, which is formed by a base member having at an underside 12 thereof, downwardly exposed bearing members 121a, 121b, 121c, 121d, 121e and an upwardly exposed face 11, which is adapted for receiving and supporting a part of a user's forearm, typically the ventral side of the user's forearm.

The wrist support 10 may be formed as a relatively flat, longitudinal member having a length of about 10-20 cm, preferably 15-20 cm, and a width of about 5-10 cm, preferably 7-10 cm.

In other embodiments, the wrist support may be designed to extend over at least 60% of the user's radius bone, as seen along the user's arm, preferably at least 70%, at least 80% or at least 90%. In some embodiments, the wrist support may extend through 100-120% of the user's radius bone. That is, the wrist support may also provide support for the user's elbow. The gaming glove may be provided in different sizes to fit differently sized users.

The wrist support 10 may be formed of a polymeric material, such as plastic or reinforced plastic. Alternatively, a relatively light metal, such as aluminum or titanium, may be used. In any event, it may be desirable to select a material that provides minimum irritation to the user's skin. In particular embodiments, the wrist support 10, and or the hand support, may be formed of a thermoplastic polyurethane or by a thermoplastic elastomeric material.

An upper surface 11 of the wrist support may present a groove running along the longitudinal direction and adapted to receive the user' forearm.

The upper surface 11 may be provided with a surface structure and/or a material that promotes transfer of air around the user's arm. For example, the surface may have protrusions and/or depressions, ridges, grooves, etc., which allow air to flow between the user's arm and the upper surface.

As an alternative, or supplement, the upper surface 11 may be provided with a textile or fibrous material.

As yet another alternative, or supplement, the upper surface 11 may present one or more padded areas, e.g. near edges of the upper surface.

As another option, at least 50%, preferably 60% or 80% of the upper surface 11 may be provided with padding to increase comfort.

Hence, the wrist support and/or the hand support may be padded, so as to provide increased comfort. Examples of material include fabrics and foamed polymer materials.

Moreover, the wrist support and/or the hand support may be formed from at least two materials, including a first material that provides rigidity and a second, less rigid material that provides comfort. For example, the first material may be a normal engineering plastic, whereas the second material may be a rubber or TPE type material. The materials may be formed separately and then bonded to each other, or they may be formed through a co-injection molding process.

A wrist fastening device 13 may be provided, configured for attaching the wrist support to the user's wrist, such that the wrist support will follow movements of the user's wrist.

The wrist fastening device 13 may comprise one or more straps, which may be resilient or non-resilient. The straps may be provided with an attachment mechanism, such as a buckle, a hook-and-loop arrangement, or the like, to allow the user to releasably and comfortably attach the wrist support to the wrist. In the case where the strap(s) is/are resilient, no attachment mechanism may be necessary.

A strap typically extends over less than 30%, preferably less than 20% or less than 10%, of the length of the wrist support.

As an alternative, a resilient cuff or wristlet may be provided, which may extend over more than 30%, preferably more than 50% or more than 80% of the length of the wrist support.

As yet another alternative, it is possible to use a lacing arrangement, similar to a shoe-lacing arrangement, but preferably adapted so as to enable it to be operated with one hand. An example of such an arrangement is disclosed in US2004172853A.

A more general example may be where the free ends of the lacing arrangements are twisted about a spool, followed by a locking action of the spool and the lace ends. For example, the spool may be axially movable between a first position, wherein it is rotatable for winding/unwinding the lace ends and a second position, wherein it is prevented from rotating. A bayonet type locking mechanism may be provided for locking the spool in the second position.

In embodiments using a sleeve or cuff, whether laced or otherwise possible to tension around the user's arm, it is possible to arrange such sleeve or cuff to be detachably attachable to the wrist support and optionally the hand support. This may be rendered possible by attaching the sleeve or cuff to the wrist/hand support by means of e.g. buttons, hook-and-look type fasteners or the like.

This may be particularly advantageous in embodiments where the sleeve or cuff extends over more than 30% of a length of the user's radius bone, such as 50-120%, preferably 50-100% of the length.

Needless to say, the sleeve or cuff may be fixedly attached to the wrist support and optionally also to the hand support.

The underside 12 of the wrist support may present the bearing members 121a, 121b, 121c, 121d, 121e. There may be 3-10, preferably 3-7 or 4-5 bearing members 121a, 121b, 121c, 121d, 121e, arranged to define a two-dimensional support surface. For example, as illustrated, four bearings 121a, 121b, 121c, 121d may be arranged at corners of the wrist support, such that they together define a rectangle, and one or more bearings 121e may be arranged inside the rectangle, e.g. at or near its geometric center of gravity.

The bearing members 121a, 121b, 121c, 121d, 121e may be ball transfer units, or similar, each comprising a housing and a load ball, and optionally a plurality of support balls. The load ball may be made of a metallic, polymeric or ceramic material.

Alternatively, or as a supplement, the underside of the wrist support may be provided with one or more low friction surfaces, e.g. surfaces coated with a low-friction type polymer, such as PTFE. Such low friction surface may extend over all or parts of the length of the wrist support and the hand support.

At least one of the housings of the ball transfer units may be received in recesses in the underside of the wrist support.

Alternatively, at least one of the housings may be arranged on the outside of the underside of the wrist support.

As yet another option, at least one of the housings may be integrated with the wrist support. For example, a recess in the underside of the wrist support may directly receive the load ball and any support balls.

The gaming glove further comprises a hand support 20. The hand support may be made of the same material as the wrist support 10.

The hand support 10 should be configured to provide support for the carpus bones of the hand, while preferably not impeding movement of the metacarpal bones.

Hence the hand support 10 may be entirely comprised within the two-dimensional footprint provided by the wrist support 10.

Alternatively, the hand support 10 may extend slightly beyond the footprint of the wrist support. Typically, such extent will be less than 20% of the length of the wrist support, preferably less than 10% or less than 5%.

An upper surface 21 of the hand support may be approximately flush with the upper surface 11 of the wrist support. To this end, the part of the wrist support 10 where the hand support 20 is connected may be recessed, so as to fit the hand support 20 and the bearing 26.

At least 50%, preferably 60% or 80% of the upper surface 21 may be provided with padding to increase comfort.

The hand support 20 may be movable relative to an upwardly facing surface of the wrist support 10. In particular, the hand support may be movable relative to the wrist support along a plane that is horizontal +/−40 degrees, preferably +/−20 degrees or +/−10 degrees. Alternatively, an interface surface between the hand support and the wrist support may be part spherical.

As one example, the hand support 20 may be rotatably connected to the wrist support 10. The rotatable connection may be configured such that the hand support and the wrist support are rotatable relative to each other about an axis A that provides an angle of less than 40 degrees, preferably less than 30 degrees or less than 15 degrees, relative to a vertical direction, when the device is placed on a horizontal surface. Typically, the axis may be substantially vertical.

The axis A may be situated within the two-dimensional footprint of the wrist support, and preferably also within a two-dimensional footprint defined by the bearings 121a, 121b, 121c, 121d on which the wrist support 10 rests on the base, e.g. a table.

The rotational connection 26 may typically comprise a roller bearing, one race of which being connected to the wrist support 10 and the other race of which being connected to the hand support 20.

Alternatively, the hand support 20 may be slidable relative to the wrist support 10. The underside 27 of the hand support and the upwardly oriented bearing surface of the wrist support may be provided with a bearing arrangement, such as a glide bearing or a roller bearing.

Moreover, one of the hand support 20 and the wrist support 10 may comprise one or more guide members, which may interact with corresponding guide members on the other one of the hand support 20 and the wrist support 10. For example, a guide member may comprise a pin or a fin and a corresponding guide member may comprise a groove or a slot, which may be curved.

The guide members may control the movement direction and limit the movement, e.g. such that the hand support 20 may move and/or rotate horizontally through a limited angle relative to the wrist support 10.

Optionally, such guide members may also limit movement in the vertical direction.

Guide members as described above may be combined with the rotational connection 26 described above.

The hand support 20 may further comprise a frame 22, 23, 24, 25, which is adapted to interact laterally with the user's hand, to make sure that any sideways movement of the hand relative to the user's forearm results in a rotation about the axis A.

As illustrated, the frame may comprise one, or a pair of, vertical section(s) 22, extending upwardly from an upper surface of the hand support 20. The frame may further comprise one or two lateral sections 24, which may extend forwardly of the vertical section 22, and optionally a transversal section 25, which may extend between distal parts of the vertical sections 24 and/or between distal parts of the lateral sections 24.

The hand support 20 may further comprise a hand fastening device 30, adapted to safely attach the user's hand to the hand support 20, such that the hand support 20 will follow the user's hand movements.

The frame 22, 23, 24, 25 may be at least partially detachably connected to the hand support 20. For example, the frame may, at one lateral side thereof, be provided with a hinge and at the other lateral side thereof with a connector. Alternatively, the frame 22, 23, 24, 25 may be so flexible as to render any hinge unnecessary.

As yet another alternative, the frame 22, 23, 24, 25 may be resilient and flexible to such an extent that it returns to a closed position after having been opened, but does not need any connector.

The hand fastening device may comprise one or more of a cuff, a wristlet, a strap or a glove. In the case of a glove, possibly a fingertip-less g love.

The hand fastening device 30 may be attached to the hand support and/or to the frame 22, 23, 24, 25, if any. For example, a hand fastening device in the form of a cuff, wristlet or glove may be attached both to the hand support 20 and to the frame 22, 23, 24, 25. It may be suitable to attach such hand support to distal parts of the lateral frame members 24, to distal parts of the vertical members 22 and/or to a cross member 23, 25.

The device may be used by arranging a lower portion of a ventral side of the user's forearm to bear against the upper side 11 of the wrist support and arranging a portion of the user's hand comprising the carpus bones to bear against an upper side of the hand support.

Hence, in use, the lower part of the user's forearm rests against the wrist support and the carpus bones of the user's hand rests against the hand support.

The wrist support may be attached to the user's forearm by means of the wrist fastening device 13.

The hand support may be attached to the user's hand by means of the hand fastening device 30.

Hence, in use, the device may be securely attached to the user's arm such that the wrist support's 10 downwardly exposed bearings reduce friction against the supporting surface, which may be a table, optionally provide with a mouse mat, thus allowing the device to follow the user's arm movements.

The hand support may be securely attached to the user's hand, such that movements of the user's hand relative to the wrist are facilitated by the bearing 26 forming the rotatable connection between the hand support 20 and the wrist support 10.

The hand frame 22, 23, 24, 25 will abut, in particular, sides of the user's hand to ensure that the user's hand movements are properly transferred to the hand support.

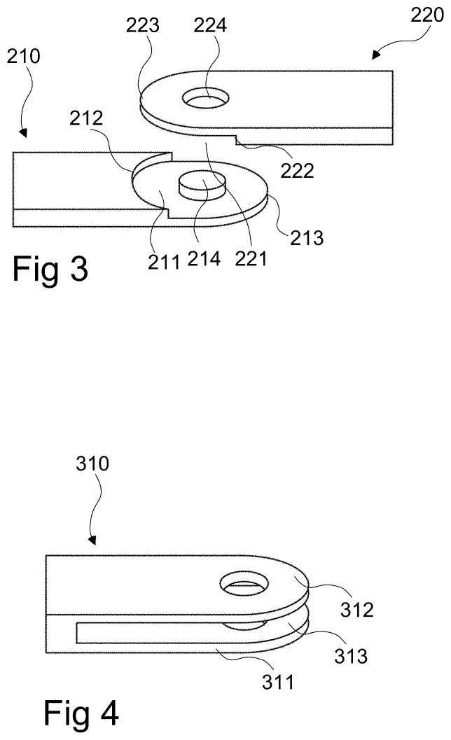

FIG. 3 schematically illustrates a first joint concept for providing a rotatable joint between the wrist support and the hand support. The joint may comprise a pair of joint members 210, 220, which may be attached to, or formed in one piece with, a respective one of the wrist support and the hand support.

The joint members may be formed so as to partially overlap each other. An area or overlap may be recessed in one or both of the joint members, such that the joint members will take up less space in the direction parallel with an axis of rotation of the joint. For example, the recesses 211, 221 may be designed so that a total thickness of the joint members at the area of overlap is about the same as that of the joint members outside the area of overlap. The difference may preferably be less than 20%, less than 10% or less than 5% of the thickness of the area outside the area of overlap.

Moreover, one or more rotation guide portions 212, 213, 214; 222, 223, 224 may be provided. In the illustrated example, there is provided an outer guide portion 212, 213; 222, 223 and a central guide portion 214; 224.

The central guide portion 214, 224 may be formed by a protrusion 214 in one of the recesses 211 and a depression or through hole 224 in the other one of the recesses 221.

The outer guide portion may be formed by an arched portion 212, 222 of the transition between the recess 211, 221 and the non-overlapping portion of the joint member. This arched portion may be formed so as to fit with a distal portion 213, 223 of the other one of the joint members.

A joint lock (not shown) may be provided with a view to preventing the joint members from separating in the direction along the axis of rotation. Such joint lock may be provided by e.g. a fastener attaching a washer to the protrusion, the washer being larger than the corresponding recess of the other joint member.

FIG. 4 schematically illustrates a second joint concept. This concept resembles the one in FIG. 3, but with one of the members 310 providing a slot 313 between a pair of edge portions 311, 312 and the other one (not shown) providing a tongue that is to be received in the slot, together with a pin or other joint lock (not shown) that may provide an axle about which the other joint member (not shown) may rotate.

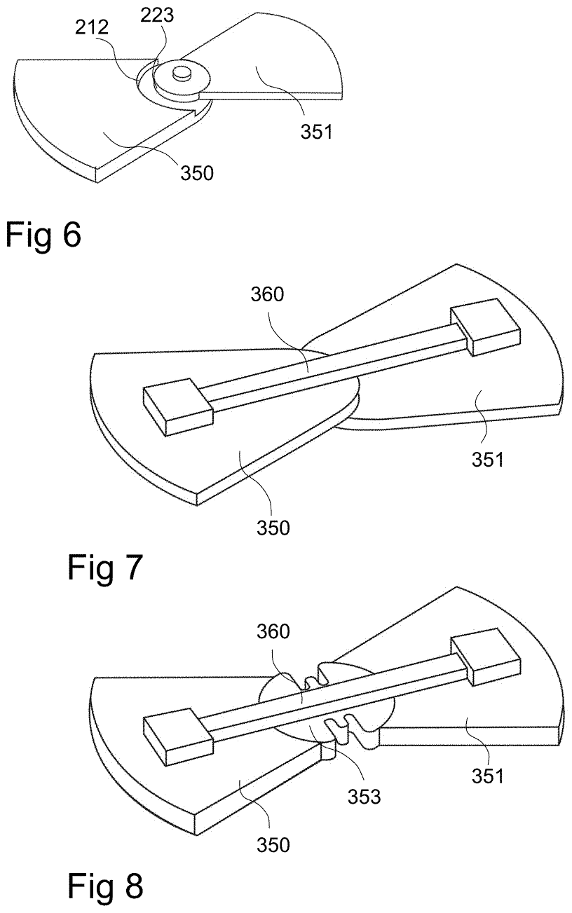

FIG. 6 illustrates a further design, wherein the arched portion 212 of one of the joint members is further spaced from the distal portion 223 of the other joint member, such that a gap is formed therebetween. This gap may correspond to at least 1%, preferably 5%, 10% or 15%, of a length of the overlap area as seen along the direction of the user's arm. The gap may reduce friction.

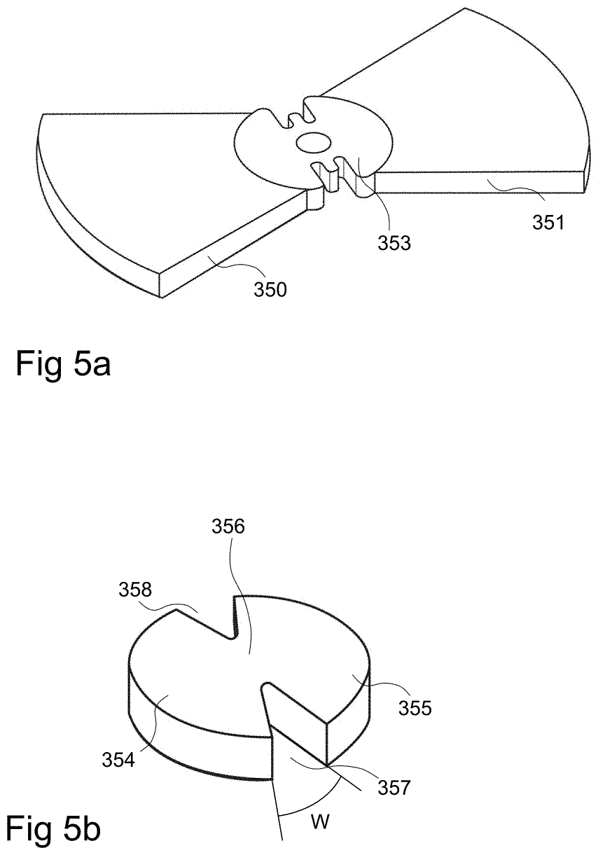

FIGS. 5a-5b schematically illustrates a third joint concept. This concept is based on a flexible member 535, which may be formed by a separate part that is attached to one or both of the hand support 351 and the wrist support 350, or which may be formed in one piece with one or both of the hand support and the wrist support.

The flexible member 353 may comprise a pair of attachment portions 354, 355, which are separated by a waist portion 356. By designing the member so that its thickness, at and near the waist portion is greater than its width at the waist portion, the member will be prone to bending about the waist portion 356.

The waist portion 356 may be formed by a respective lateral space 357, 358 being provided to extend from an outer lateral edge of the member and inwardly towards the waist portion 356. The lateral spaces 357, 358 may be designed to define a respective central angle W about a bending axis, that will limit the lateral bending of the member.

It is thus possible to control the amount of bending that is to be allowed through the size of the lateral space. Notably, it is possible to provide symmetric or asymmetric bending properties.

With a flexible member, it is possible to provide a bias towards a predetermined relative position of the hand support and the wrist support, in that the flexible member may be elastically flexible, so as to be prone to returning to its original, unloaded position.

The flexible member may be formed of an elastomeric material, which may be foamed. Examples of materials include rubber materials, polyurethane and thermoplastic elastomers.

The flexible member may be releasably attachable to the joint members, such that it is possible to exchange it, e.g. in order to change the angle limitations and/or to change the stiffness of the joint.

FIG. 7 and FIG. 8 illustrate embodiments wherein such a biased effect can be provided by means of a spring member 360. The spring member 360 may be combined with the articulated joint designs according to FIGS. 3, 4 and 6. Moreover, the spring member 360 may be used together with the flexible and optionally elastic member 353 of FIGS. 5a-5b, as illustrated in FIG. 8.

The spring member 360 may be releasably attached, such that it is possible to change it in order to provide a desired stiffness of the joint.

Finally, and as illustrated in FIG. 7, it is possible to base the entire joint on the spring member 360. That is, the spring member may be the device which holds the joint members together. Optionally, an area of overlap between the joint members may be used to limit the bending motion to a single plane.

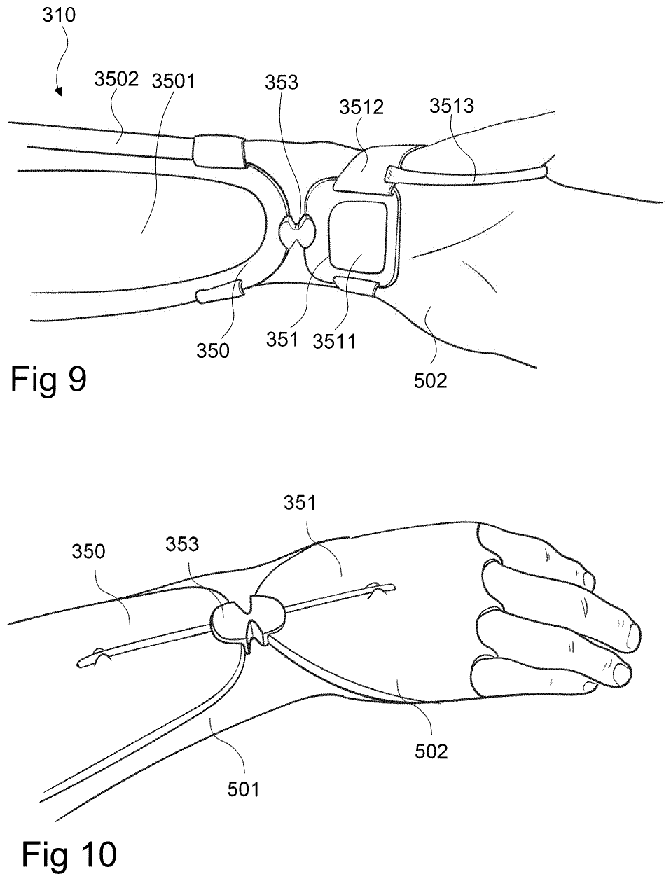

FIG. 9 schematically illustrates an embodiment of a gaming glove worn by a user, as seen from below. In this figure, the wrist support and the hand support are illustrated, as is the joint. While in the illustrated version, the joint is based on FIGS. 5a-5b, it is understood that any one of the joint designs provided herein may be used.

Moreover, in FIG. 9, there is illustrated the bearing arrangements 3501, 3511 which are provided on the wrist support 350 and on the hand support 351, as well as the sleeves 3502, 3512 and straps 3513 that are used to attach the glove to the user's lower arm, to his hand and to his fingers. It is noted that in the embodiment illustrated in FIG. 9, the joint is provided on the underside of the device, i.e. below the user's arm and closer to the support surface on which the computer mouse is operated.

FIG. 10 schematically illustrates an embodiment of a gaming glove worn by a user, as seen from above. In this embodiment, the joint 353 is arranged on the upper part of the glove, i.e. on the upper side of the user's hand 502 and wrist 501. The joint may be designed just like in FIG. 9.

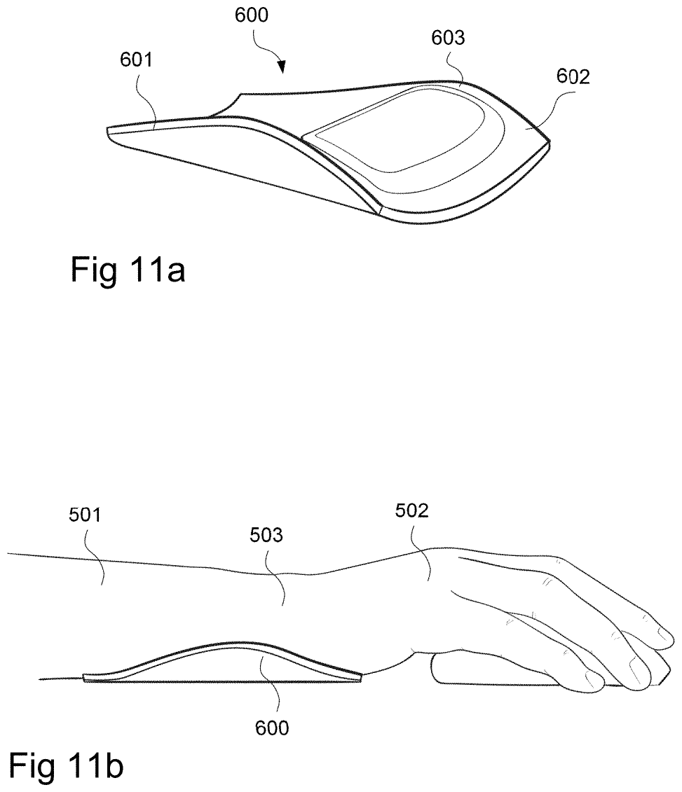

FIGS. 11a-11b schematically illustrate an ergonomic support member 600, which may be incorporated into the gaming glove. This ergonomic support member is designed to insure that the distal part of the user's radius bone is spaced further from the support surface than it would normally be, such that a desired angle of the user's wrist can be effortlessly maintained.

The support member may comprise a first support surface 601 that is straight or slightly curved in the longitudinal direction of the user's arm and concave as seen in a transversal direction. This first support surface may be intended to support part of the user's lower arm 501. The support member may comprise a summit portion 603, which is concave also in the longitudinal direction, where the user's wrist joint 503 is to be positioned. Finally, the support member may comprise a second support surface 602, that is intended to support the proximal portion of the user's hand 502. When placed on a horizontal surface, the second support surface may present a greater slope than the first support surface, as seen along the longitudinal direction of the user's arm.

The support member 600 may be integrated, such as attached to or formed in one piece with, the wrist support.

The support member 600 may present one or more through holes or openings, such that it will allow air flow to/from the user's arm.

While the device may be used with any type of computer mouse and with any type of program, one specifically suitable use case is for first-person-shooter type games, such as Counter-Strike: Global Offensive®. In such use, a mouse sensitivity may typically be set to less than about 1000 DPI (dots per inch). In such use, the wrist support will be essential in providing friction reduction for large sideways movements of the mouse.

Another specifically suitable use case is for the multiplayer online battle arena type games, such as League Of Legends®. In such use, a mouse sensitivity may typically be set to more than about 2000 DPI (dots per inch). In such use, the hand support will be essential in providing friction reduction for small sideways movements of the mouse.

It may be desirable to provide one or more joint covers. For example, referring to FIG. 9, it may be desirable to provide a joint cover on the underside of the joint, to provide a smooth transition between the wrist support 350 and the hand support 351. Such a support may be formed by extending the hand support and the wrist support such that they provide a rotary joint with closely spaced arched edges like illustrated in FIG. 4, as opposed to the open joint illustrated in FIG. 9. Alternatively, a portion of the hand support and/or of the wrist support may extend below the joint, and optionally overlap the other one of the hand support and/or the wrist support.

For example, it may be desirable to provide a downwardly facing support which extends all the way from the hand support through the wrist support to provide a substantially continuous downwardly facing support surface. Such downwardly facing support surface may thus be continuous as a consequence of an overlap, or it may be articulated.

It may also be desirable to provide padding on the side of the joint that faces the user's wrist joint.

In a further embodiment, the gaming glove may comprise a sleeve that is adapted to provide a compression effect on the user's wrist and/or forearm. In particular, such sleeve may be adapted to provide a compression on the order of 15-60 mmHG. In various embodiments, the sleeve may be designed to provide a pressures in the following ranges: 20-30 mmHg, 30-40 mmHg, 40-50 mmHg or 50-60 mmHg. In yet further embodiments, the sleeve may be designed to provide pressures in the following ranges: 15-21 mmHg, 23-32 mmHg or 34-46 mmHg.

The sleeve may be provided as a closed sleeve that is pulled onto the user's arm like a sock. Alternatively, the sleeve may be at least partially open, along its length, such that it can be closed by e.g. a zipper, hook-and-loop type fastener or the like.

The sleeve may separate from the remainder of the gaming glove, such that the sleeve is applied to the user's wrist or forearm prior to applying the glove. Alternatively, the sleeve may be attached to or attachable to, the gaming glove.

The sleeve present a length and shape sufficient for it to extend from above the user's elbow, along the forearm and over part of the user's hand. In particular, the sleeve may present a loop, strap or hole adapted for receiving at least one of the user's fingers, for example the user's thumb.

Moreover, at the portion of the gaming glove overlapping the user's hand, it may be desirable to leave the underside of the hand uncovered, such that direct skin contact with the computer mouse is achieved.

Such longer sleeves may be particularly preferred in conjunction with a wrist support designed to extend over at least 60% of the user's radius bone, as seen along the user's arm, preferably at least 70%, at least 80% or at least 90%. In some embodiments, the wrist support may extend through 100-120% of the user's radius bone.

Where a separate sleeve is used, the wrist fastening device and the hand fastening device may operate as described above.

As yet another feature, it may be possible to provide an illumination device in or on the gaming glove. For example, an illumination device may be provided to illuminate the base on which the gaming glove/mouse are operated. Such illumination device may comprise a power source, such as a battery, one or more light sources, such as LEDs and optionally one or more light guides. A controller may also be provided, e.g. to turn the illumination on/off and optionally also to change color of the illumination, should the light sources be provided in the form of e.g. RGB enable light sources. The controller may communicate with e.g. a computer, a computer peripheral or a smartphone/tablet. Such communication may be wireless. Thus a user interface may be provided via an app in the smartphone/tablet or in the computer.

1. A device for increasing performance, comfort and/or preventing injury of a user of a computer mouse or the like, comprising:
a wrist support, having an upper side adapted for receiving the user's wrist and/or lower forearm and an underside adapted to bear against a planar surface, such as a table,
a hand support, adapted for receiving a part of the user's hand, and a wrist fastening device, for securing the wrist to the wrist support, wherein the underside presents a bearing arrangement for reducing friction against the planar surface, and
wherein the hand support is horizontally +/−40 degrees, movable relative to the wrist support.

2. The device as claimed in claim 1, wherein the hand support is movably connected to the wrist support.

3. The device as claimed in claim 1, wherein movement between the hand support and the wrist support is limited.

4. (canceled)

5. The device as claimed in claim 1, wherein the hand support is movably connected to the wrist support by means of a connection, which comprises a rotatable connection by which the hand support is rotatably connected to the wrist support, such that the hand support and the wrist support are rotatable relative to each other about an axis (A) that provides an angle of less than 40 degrees relative to a vertical direction, when the device is placed on a horizontal surface.

6. The device as claimed in claim 1, wherein the hand support is connected to the wrist support by a flexible element, which is fixedly connected to each of the hand support and the wrist support.

7. The device as claimed in claim 6, wherein the flexible element comprises a pair of attachment portions, connected to a respective one of the hand support and the wrist support and a waist portion, whereby the attachment portions are movable relative to each other through bending about the waist portion.

8. (canceled)

9. The device as claimed in claim 1, wherein a biasing element is provided to bias the hand support and the wrist support towards a predetermined relative position.

10. The device as claimed in claim 1, wherein the hand support is connected to the wrist support by means of a joint which is provided at a position below the user's hand.

11-13. (canceled)

14. The device as claimed in claim 1, wherein the bearing arrangement comprises a friction reducing surface, arranged on the underside of the wrist support, and optionally also on an underside of the hand support.

15-16. (canceled)

17. The device as claimed in claim 1, wherein the wrist fastening device comprises one or more straps, a cuff or a wristlet.

18. The device as claimed in claim 1, further comprising a hand frame, connected to the hand support, extending upwardly and/or forwardly and configured to at least laterally interact with the hand.

19. The device as claimed in claim 1, further comprising a hand fastening device for securing the hand to the hand support.

20. (canceled)

21. The device as claimed in claim 1, wherein the wrist support is sized to extend over at least 60% of the user's radius bone.

22. The device as claimed in claim 1, further including a wrist positioning element (404 comprising a support surface that is shaped so as to position a distal portion of the user's radius bone at a higher vertical level than a proximal portion of the user's radius bone.

23. A kit of parts comprising:
a device as claimed in claim 1, and
a sleeve, adapted for being applied to a wrist or forearm of the user.

24. The kit as claimed in claim 23, wherein the sleeve is a compression sleeve, adapted to provide a compressive pressure of 15-60 mmHg on at least a portion of the user's wrist or forearm.

25. The kit as claimed in claim 23, wherein the sleeve is attached or attachable to the device.

26. A method of applying a device for increasing performance, comfort and/or preventing injury of a user of a computer mouse or the like, comprising:
providing a device as claimed in claim 1,
arranging a lower portion of a ventral side of the user's wrist and/or lower forearm to bear against the upper side of the wrist support, and
arranging a portion of the user's hand comprising the carpus bones to bear against an upper side (21) of the hand support.
attaching the user's wrist and/or lower forearm to the wrist support by means of a wrist fastening device, and
optionally attaching the user's hand to the hand support by means of a hand fastening device.

27. (canceled)

28. The method as claimed in claim 26, further comprising applying a compression sleeve to the user's wrist or forearm prior to arranging the lower portion of the ventral side of the user's wrist to bear on the upper side of the wrist support.

29. (canceled)

30. The device as claimed in claim 14, wherein the bearing arrangement additionally comprises a friction reducing surface arranged on an underside of the hand support.
